(19) United States
(12) Patent Application Publication
(10) Pub. No.: US 2024/0274261 A1
(43) Pub. Date: Aug. 15, 2024

(54) SYSTEM, DEVICE, AND METHOD FOR CONTROLLING REHABILITATION OF PATIENT

(71) Applicant: ELECTRONICS AND TELECOMMUNICATIONS RESEARCH INSTITUTE, Daejeon (KR)

(72) Inventors: Su Ran PARK, Daejeon (KR); Hang Kee KIM, Daejeon (KR); Ki Suk LEE, Daejeon (KR); Dong Chun LEE, Daejeon (KR)

(73) Assignee: ELECTRONICS AND TELECOMMUNICATIONS RESEARCH INSTITUTE, Daejeon (KR)

(21) Appl. No.: 18/436,170

(22) Filed: Feb. 8, 2024

(30) Foreign Application Priority Data
Feb. 10, 2023 (KR) 10-2023-0017946

(51) Int. Cl. G16H 20/30; G06T 7/00; G06T 17/00; G16H 10/60

(52) U.S. Cl. CPC G16H 20/30; G06T 7/00; G06T 17/00; G16H 10/60; G06T 2207/10021

(57) ABSTRACT

Provided are a system, device, and method for controlling rehabilitation of a patient. The system includes a first device configured to create a virtual model for a first user on the basis of physical information of the first user using digital twin technology and a second device configured to visualize the virtual model and provide the visualized virtual model to a second user.

### CROSS-REFERENCE TO RELATED APPLICATION

This application claims priority to and the benefit of Korean Patent Application No. 10-2023-0017946, filed on Feb. 10, 2023, the disclosure of which is incorporated herein by reference in its entirety.

### BACKGROUND

### 1. Field of the Invention

The present invention relates to a system, device, and method for controlling rehabilitation of a patient.

### 2. Discussion of Related Art

Digital twins, a concept advocated by General Electric Company of the US, correspond to a technology for creating a computerized twin of a real-world object and simulating situations that may occur in the real world to predict outcomes in advance. These digital twins have primarily been applied to manufacturing. These days, however, digital twins are attracting attention as a technology for solving a variety of industrial and social problems, and the scope of application is continuously expanding in combination with the Internet of things (IoT), augmented reality (AR) technology, artificial intelligence (AI) technology, and the like. In the field of medicine, digital twin-based medical services are being used in various ways for patient monitoring and the like.

Meanwhile, people with brain disorders (injuries) have difficulty living at home because their motor skills are degraded due to the damaged central nervous system of the brain, and they may experience accidents such as falling and the like. Continuous rehabilitation training is necessary to restore the motor function of patients with these brain disorders. However, according to the related art, patients are required to be hospitalized or visit a hospital for rehabilitation training, and this limitation makes it difficult for patients to perform rehabilitation training.

These days, healthcare services have emerged that combine rehabilitation training with augmented reality (AR) or virtual reality (VR)-based game content so that rehabilitation training may be performed at home. However, these healthcare services are performed by patients or caregivers without any medical knowledge, resulting in ineffective rehabilitation and risk of injury.

### SUMMARY OF THE INVENTION

The present invention is directed to providing a system, device, and method for controlling rehabilitation of a patient which allow a patient's information related to usage of rehabilitation training content to be shared with a medical professional so that a diagnosis or prescription for the patient may be made remotely.

According to an aspect of the present invention, there is provided a system for controlling rehabilitation of a patient, the system including a first device configured to create a virtual model for a first user on the basis of physical information of the first user using digital twin technology and a second device configured to visualize the virtual model and provide the visualized virtual model to a second user.

The first device may provide rehabilitation training content to the first user and collect usage information of the first user for the rehabilitation training content.

The usage information may include at least one of video information of the first user, sensing information of the first user, and result information of the rehabilitation training content.

The second device may provide the usage information or information obtained by processing the usage information to the second user.

The second device may generate a stereoscopic video by applying the usage information to the virtual model and provide the stereoscopic video to the second user.

The second device may receive setting information for the rehabilitation training content from the second user, perform a kinematic simulation of causing the virtual model to perform the rehabilitation training content to which the received setting information is applied, and provide at least one of a stereoscopic video and a result of the simulation to the second user.

The second device may receive final setting information for the rehabilitation training content from the second user and update setting information for the rehabilitation training content provided to the first user according to the final setting information.

According to another aspect of the present invention, there is provided a method of controlling rehabilitation of a patient which is performed by a patient rehabilitation control system, the method including creating a virtual model for a first user on the basis of physical information of the first user using digital twin technology and visualizing the virtual model and providing the visualized virtual model to a second user.

According to another aspect of the present invention, there is provided a device for controlling rehabilitation of a patient, the device including an output module and a processor connected to the output module. The processor creates a virtual model for a patient on the basis of physical information of the patient using digital twin technology, visualizes the virtual model, and provides the visualized virtual model to a user.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram of a system for controlling rehabilitation of a patient according to an exemplary embodiment of the present invention;

FIG. 2 is a conceptual diagram of a system for controlling rehabilitation of a patient according to an exemplary embodiment of the present invention;

FIG. 3 is an illustration of an operational scenario of a system for controlling rehabilitation of a patient according to an exemplary embodiment of the present invention;

FIG. 4 is an illustration of a user interface of a rehabilitation control application according to an exemplary embodiment of the present invention;

FIG. 5 is an illustration of a medical data dashboard according to an exemplary embodiment of the present invention;

FIG. 6 is a block diagram of a rehabilitation training provision device according to an exemplary embodiment of the present invention;

FIG. 7 is a block diagram of a rehabilitation training control device according to an exemplary embodiment of the present invention;

FIG. 8 is a first sequence diagram illustrating a rehabilitation training control method according to an exemplary embodiment of the present invention;

FIG. 9 is a second sequence diagram illustrating a rehabilitation training control method according to an exemplary embodiment of the present invention; and

FIG. 10 is a third sequence diagram illustrating a rehabilitation training control method according to an exemplary embodiment of the present invention.

### DETAILED DESCRIPTION OF EXEMPLARY EMBODIMENTS

A system, device, and method for controlling rehabilitation of a patient according to exemplary embodiments of the present invention will be described below. In this process, the thicknesses of lines, the sizes of components, and the like shown in the drawings may be exaggerated for the purpose of clarity and convenience of description. Also, terms to be described below are defined in consideration of functions in the present invention, and the terms may vary depending on the intention of a user or operator or precedents. Therefore, these terms are to be defined on the basis of the overall content of the specification.

Hereinafter, exemplary embodiments of the present disclosure will be described in detail with reference to the accompanying drawings so that those of ordinary skill in the art may easily implement the present invention. However, the present invention may be implemented in a variety of different forms and is not limited to the exemplary embodiments described herein. To clearly describe the present invention, elements irrelevant to description will be omitted in the drawings, and like reference numerals refer to like elements throughout the drawings.

As used herein, when a first component is described as “including” or “having” a second component, other components are not precluded but may be further included unless particularly described otherwise.

The following description may be implemented, for example, as a method, a process, a device, a software program, a data stream, or a signal. Although discussed only in the context of a single form of implementation (e.g., only as a method), discussed features may also be implemented in other forms (e.g., as a device or program). The device may be implemented using appropriate hardware, software, firmware, and the like. The method may be implemented in a device such as a processor or the like which generally refers to a processing device including a computer, a microprocessor, an integrated circuit, a programmable logic device, or the like.

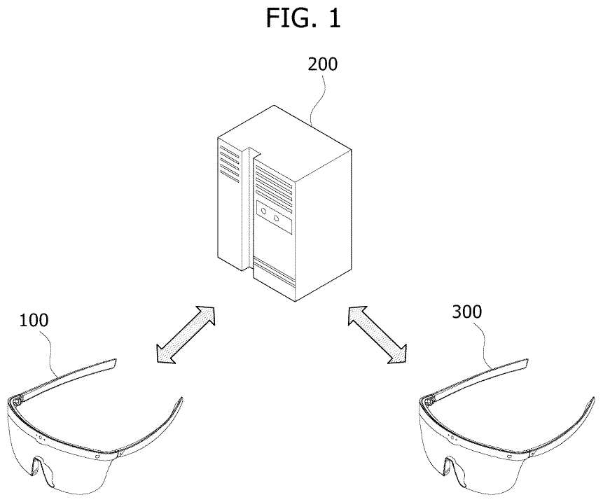

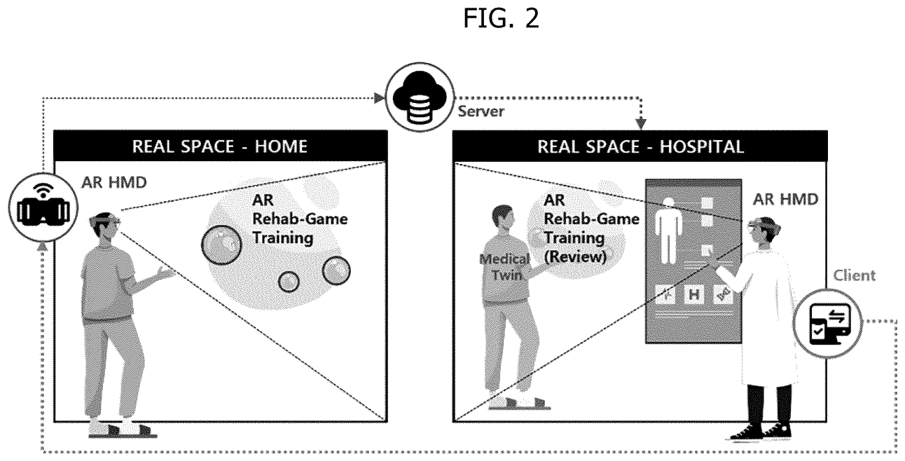

FIG. 1 is a block diagram of a system for controlling rehabilitation of a patient according to an exemplary embodiment of the present invention, and FIG. 2 is a conceptual diagram of a system for controlling rehabilitation of a patient according to an exemplary embodiment of the present invention.

Referring to FIGS. 1 and 2, a system for controlling rehabilitation of a patient according to the exemplary embodiment of the present invention may include a first device 100, a server 200, and a second device 300.

The first device 100 is a device used by a first user (e.g., a patient) and may be a device for supporting augmented reality (AR) technology (e.g., HoloLens2 of Microsoft). The first device 100 may provide rehabilitation training content to the first user and collect usage information of the first user for the rehabilitation training content. Here, the rehabilitation training content may be game content that instructs the first user to move a part or all of his or her body to a specific position or in a specific manner.

The rehabilitation training content may be classified as, but is not limited to, upper extremity rehabilitation content, full-body rehabilitation content, or walking-full body rehabilitation content depending on a body area being trained. The type and intensity of rehabilitation training content provided to the first user through the first device 100 may be set by the first user through the first device 100 or set by a second user (e.g., a medical professional) through the second device 300.

The usage information of the first user for the rehabilitation training content may include at least one of video information of the first user, sensing information of the first user, and result information of the rehabilitation training content (information on results of performing the rehabilitation training content). The first device 100 may collect the video information of the first user by imaging the first user who is using the rehabilitation training content through an embedded camera. Also, the first device 100 may collect the sensing information of the first user by detecting motion of the first user who is using the rehabilitation training content through an embedded sensor.

The first device 100 may create a virtual model for the first user on the basis of physical information of the first user using digital twin technology. The first device 100 may generate an avatar based on digital twin technology from physical information of the first user. The first device 100 may receive the physical information of the first user from the first user or the outside thereof (e.g., the server 200).

The first device 100 may communicate with the server 200 through a communication network and transmit information on the virtual model (hereinafter “virtual model information”) of the first user to the server 200 through the communication network. Also, the first device 100 may transmit the usage information of the first user for the rehabilitation training content to the server 200 through the communication network.

The server 200 may receive and store the virtual model information of the first user and the usage information of the first user for the rehabilitation training content transmitted from the first device 100. Also, personal information (e.g., name, age, and the like) and health information (e.g., disease name, condition, diagnosis status, and the like) of the first user may be stored in the server 200 in advance.

The second device 300 is a device used by the second user (e.g., a medical professional) and may be a device for supporting AR technology (e.g., HoloLens2 of Microsoft). The second device 300 may visualize at least one of the virtual model information of the first user, the usage information of the first user for the rehabilitation training content, the personal information of the first user, and the health information of the first user and provide the visualized information to the second user. For example, the second device 300 may implement the avatar of the first user generated using digital twin technology through AR technology and provide the avatar to the second user. Also, the second device 300 may provide the second user with a video of the first user utilizing the rehabilitation training content through AR technology. Further, the second device 300 may provide the second user with the information on results of performing the rehabilitation training content, the personal information of the first user, and the like through AR technology.

The second device 300 may generate a stereoscopic video by applying the usage information of the first user for the rehabilitation training content to the virtual model for the first user and provide the generated stereoscopic video to the second user. The second device 300 may provide the second user with a three-dimensional (3D) stereoscopic video of the first user engaging with the rehabilitation training content using the virtual model of the first user. Also, the second device 300 may generate a stereoscopic video by applying preset motion information (e.g., motion facilitating checking of the patient's condition) to the virtual model of the first user and provide the generated stereoscopic video to the second user. In this way, according to the present invention, a video of a patient related to rehabilitation training is provided to a medical professional so that the medical professional can make a diagnosis or prescription remotely.

The second device 300 may receive setting information for the rehabilitation training content from the second user, perform a kinematic simulation of causing the virtual model of the first user to perform the rehabilitation training content to which the received setting information is applied, and provide at least one of a stereoscopic video and a result of the simulation to the second user. Here, the setting information for the rehabilitation training content may include information on a type of rehabilitation training (a type of exercise), intensity of the rehabilitation training (the number of repetitions, intervals, duration, and the like), and the like. In other words, the second user can previously check load, rehabilitation training effects, and the like exerted on the first user's body when a specific rehabilitation training is performed, through a simulation employing a digital twin-based virtual model and detect the most appropriate setting of rehabilitation training content for the first user by repeating such a simulation.

The second device 300 may receive final setting information for rehabilitation training content from the second user and update setting information for the rehabilitation training content provided to the first user according to the received final setting information. Here, the final setting information may be setting information for rehabilitation training content determined by the second user on the basis of the simulation results. The second device 300 may change the setting information for the rehabilitation training content provided to the first user according to the final setting information set by the second user so that more effective rehabilitation training content can be provided to the first user. The setting information for the rehabilitation training content provided to the first user may be stored in the server 200 or the first device 100, and the second device 300 may access the server 200 or the first device 100 through the communication network and change the setting information stored in the server 200 or the first device 100 to update the setting information for the rehabilitation training content provided to the first user.

When the final setting information is received from the second user, the second device 300 may generate rehabilitation training information by matching the final setting information for the rehabilitation training content, the usage information of the first user for the rehabilitation training content, the health information of the first user, and the personal information of the first user to each other and transmit the generated rehabilitation training information to the server 200, and the server 200 may store the rehabilitation training information received from the second device 300. The rehabilitation training information stored in the server 200 may be used for reference in the rehabilitation training of a patient who requires rehabilitation training similar to that of the first user or used as an education resource for training medical professionals in rehabilitation training.

In the foregoing embodiment, it has been described that the process of creating the virtual model for the first user is performed by the first device 100, but the process may also be performed by the server 200 or the second device 300. Also, in the foregoing embodiment, it has been described that the process of generating the stereoscopic video and the process of performing the simulation are performed by the second device 300, but the processes may also be performed by the server 200.

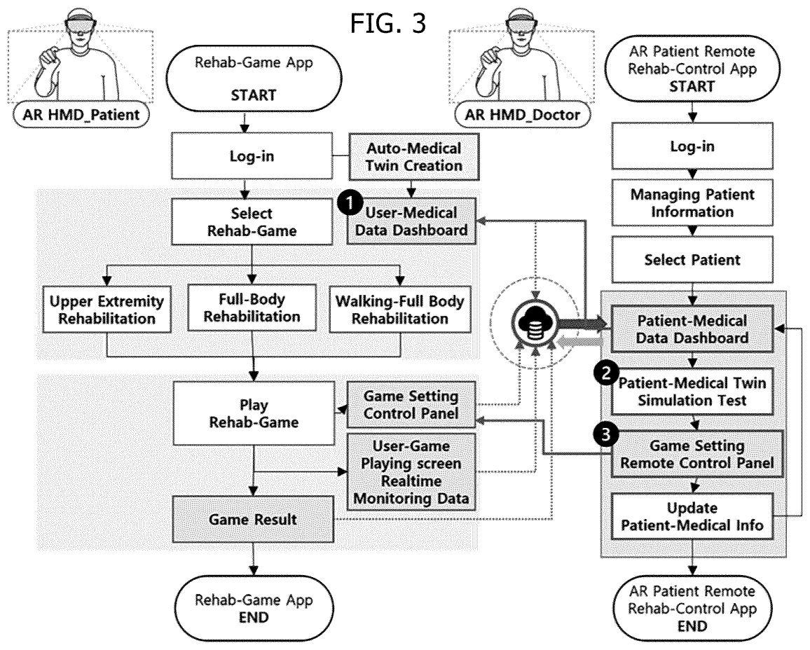

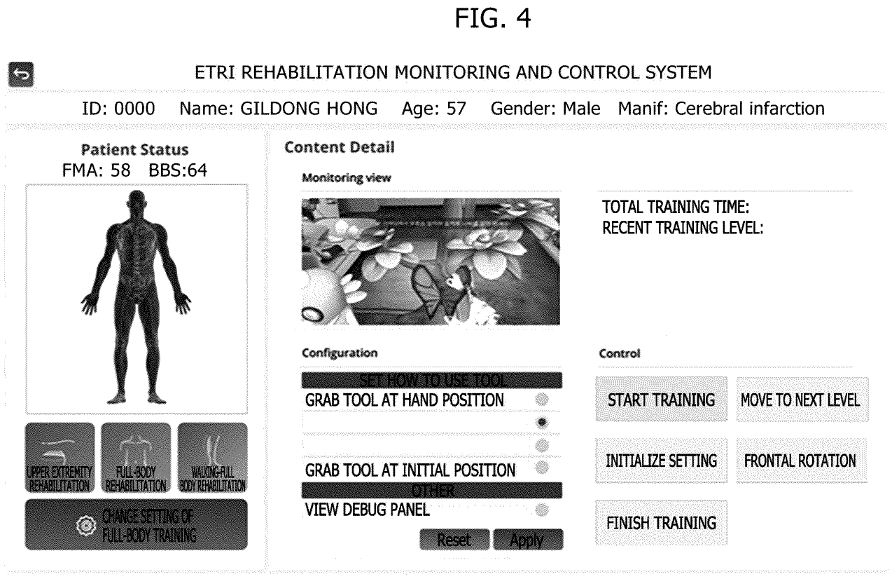

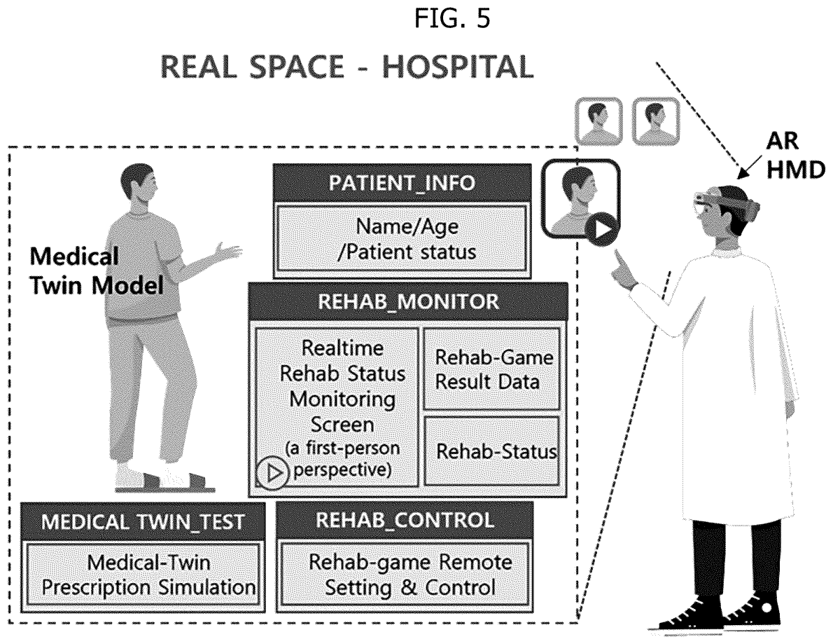

FIG. 3 is an illustration of an operational scenario of a system for controlling rehabilitation of a patient according to an exemplary embodiment of the present invention, FIG. 4 is an illustration of a user interface of a rehabilitation control application according to an exemplary embodiment of the present invention, and FIG. 5 is an illustration of a medical data dashboard according to an exemplary embodiment of the present invention.

An operational scenario of a system for controlling rehabilitation of a patient according to an exemplary embodiment of the present invention will be described below with reference to FIGS. 3 to 5.

First, a patient may execute a rehabilitation training application through the first device 100 and perform a sign-up process to set his or her username and password and input his or her physical information to the server 200. When the sign-up process has already been completed, the patient may perform a login process of inputting the set username and password. Here, the rehabilitation training application may be an application installed on the first device 100 to perform the functions of the first device 100 described above.

Subsequently, the first device 100 may create a virtual model for the patient on the basis of the physical information of the registered patient (Auto-Medical Twin Creation) and transmit information on the created virtual model (virtual model information) to the server 200 (User-Medical Data Dashboard).

Subsequently, the first device 100 may provide rehabilitation training content to the patient. For example, the first device 100 may provide one of upper extremity rehabilitation content, full-body rehabilitation content, or walking-full body rehabilitation content selected by the patient to the patient. Here, the first device 100 may collect usage information of the patient for the rehabilitation training content in real time (user-game playing screen realtime monitoring data) and transmit the collected information to the server 200.

Meanwhile, a medical professional may execute a rehabilitation control application through the second device 300 and perform a login process of inputting a set username and password. Here, the rehabilitation control application may be an application installed on the second device 300 to perform the functions of the second device 300 described above. The rehabilitation control application may provide a user interface as shown in FIG. 4 to support the functions of the second device 300 described above.

When the login is completed, the second device 300 may output information on a list of patients for whom the medical professional is responsible to the medical professional (managing patient information). In the server 200, information on patients for whom medical professionals are responsible may be stored in advance, and the second device 300 may detect the list of patients for whom the medical professional is responsible on the basis of the information stored in the server 200 and output the detected list of patients.

When a patient is selected by the medical professional, the second device 300 may output information on the selected patient. Here, the second device 300 may provide a medical data dashboard of the selected patient to the medical professional. Here, the medical data dashboard may visualize the information on the patient using AR technology.

As shown in FIG. 5, the medical data dashboard may include a graphic panel for providing a virtual model for a patient (medical twin model), a graphic panel for providing usage information of the patient for rehabilitation training content (REHAB_MONITOR), and a graphic panel for providing personal information and health information of the patient (PATIENT_INFO). For example, the REHAB_MONITOR panel may display information on a real-time video or recorded video of the patient who is using the rehabilitation training content, information on results of the patient performing the rehabilitation training content, information on rehabilitation status, and the like.

The medical data dashboard may include a graphic panel REHAB_CONTROL for providing a function of changing setting information for the rehabilitation training content provided to the patient and a graphic panel MEDICAL TWIN_TEST for providing a function of performing a kinematic simulation for the virtual model of the patient. The REHAB_CONTROL panel and the MEDICAL TWIN_TEST panel may include a graphical user interface for supporting the foregoing functions. Meanwhile, the REHAB_CONTROL panel may further include a graphical user interface for remotely causing the first device 100 to execute the rehabilitation training content or remotely terminating the rehabilitation training content executed in the first device 100. The medical professional may remotely command execution or termination of the rehabilitation content by controlling the graphical user interface included in the REHAB_CONTROL panel.

Meanwhile, the second device 300 may perform a kinematic simulation of causing the virtual model of the patient to perform the rehabilitation training content while changing the setting information for the rehabilitation training content and provide the results to the medical professional.

Subsequently, the second device 300 may update the setting information for the rehabilitation training content provided to the patient according to final setting information for rehabilitation training content determined by the medical professional on the basis of the simulation results.

Subsequently, the second device 300 may update health information of the patient according to the final setting information or prescription information input by the medical professional.

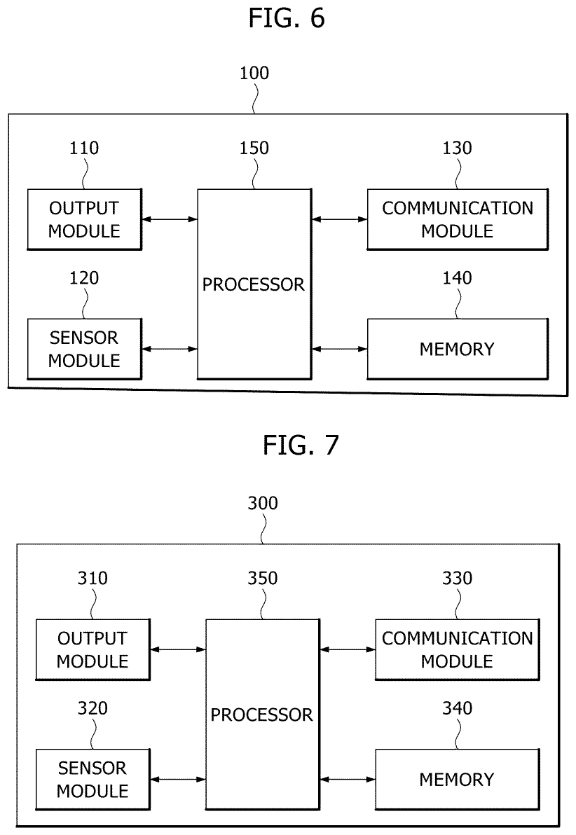

FIG. 6 is a block diagram of a rehabilitation training provision device according to an exemplary embodiment of the present invention.

Referring to FIG. 6, the rehabilitation training provision device according to the exemplary embodiment of the present invention may include an output module 110, a sensor module 120, a communication module 130, a memory 140, and a processor 150. The rehabilitation training provision device according to the exemplary embodiment of the present invention may include various components in addition to the components shown in FIG. 6, or some of the components may be omitted. The rehabilitation training provision device according to the exemplary embodiment of the present invention may correspond to the first device 100 of FIG. 1.

The output module 110 may output visual information to a user under the control of the processor 150 which will be described below. For example, the output module 110 may include a transparent display or a head-up display (HUD).

The sensor module 120 may detect the user's motion and output the detection result to the processor 150. For example, the sensor module 120 may include at least one of a camera, an acceleration sensor, an ultrasonic sensor, a lidar sensor, and a radar sensor.

The communication module 130 may communicate with an external device (e.g., the server 200). The communication module 130 may communicate with the external device according to various types of communication methods.

The memory 140 may store various types of information required for operation processes of the processor 150. Also, the memory 140 may store various types of information produced in operation processes of the processor 150.

The processor 150 may be operatively connected to the output module 110, the sensor module 120, the communication module 130, and the memory 140. The processor 150 may be implemented as a central processing unit (CPU), a microcontroller unit (MCU), or a system on chip (SoC). The processor 150 may control a plurality of hardware or software components connected thereto by running an operating system (OS) or an application, perform various types of data processing and computation, execute at least one instruction stored in the memory 140, and store the execution result data in the memory 140.

The processor 150 may provide rehabilitation training content to the user through the output module 110 and collect usage information of the user for the rehabilitation training content through the sensor module 120. Here, the processor 150 may provide the user with rehabilitation training content which is set according to setting information for rehabilitation training content stored in an external device (e.g., the server 200) or the memory 140.

The processor 150 may create a virtual model for the user on the basis of physical information of the user. The processor 150 may create the virtual model for the user from the physical information of the user stored in an external device (e.g., the server 200) or the memory 140 using digital twin technology.

The processor 150 may transmit the usage information of the user for the rehabilitation training content and information on the virtual model of the user (virtual model information) to the server 200 through the communication module 130. The processor 150 may transmit the usage information of the user for the rehabilitation training content and the virtual model information to the server 200 so that the corresponding information may be shared with a medical professional.

FIG. 7 is a block diagram of a rehabilitation training control device according to an exemplary embodiment of the present invention.

Referring to FIG. 7, the rehabilitation training control device according to the exemplary embodiment of the present invention may include an output module 310, a sensor module 320, a communication module 330, a memory 340, and a processor 350. The rehabilitation training control device according to the exemplary embodiment of the present invention may include various components in addition to the components shown in FIG. 7, or some of the components may be omitted. The rehabilitation training control device according to the exemplary embodiment of the present invention may correspond to the second device 300 of FIG. 1.

The output module 310 may output visual information to a user under the control of the processor 350 which will be described below. For example, the output module 310 may include a transparent display or an HUD.

The sensor module 320 may detect the user's motion and output the detection result to the processor 350. For example, the sensor module 320 may include at least one of a camera, an acceleration sensor, an ultrasonic sensor, a lidar sensor, and a radar sensor.

The communication module 330 may communicate with an external device (e.g., the server 200). The communication module 330 may communicate with the external device according to various types of communication methods.

The memory 340 may store various types of information required for operation processes of the processor 350. Also, the memory 340 may store various types of information produced in operation processes of the processor 350.

The processor 350 may be operatively connected to the output module 310, the sensor module 320, the communication module 330, and the memory 340. The processor 350 may be implemented as a CPU, an MCU, or an SoC. The processor 350 may control a plurality of hardware or software components connected thereto by running an OS or an application, perform various types of data processing and computation, execute at least one instruction stored in the memory 340, and store the execution result data in the memory 340.

The processor 350 may create a virtual model for a patient on the basis of physical information of the user. The processor 350 may create the virtual model for the patient from the physical information of the patient using digital twin technology. The processor 350 may receive the physical information of the patient from in an external device (e.g., the server 200) or the rehabilitation training provision device through the communication module 330. According to another embodiment, the processor 350 may receive information on the virtual model for the patient from the outside of the rehabilitation training control device.

The processor 350 may visualize the virtual model of the patient and provide the visualized virtual model to the user. The processor 350 may visualize the virtual model of the patient and provide the visualized virtual model to the user to facilitate a diagnosis or prescription for the patient.

The processor 350 may receive usage information of the patient for rehabilitation training content through the communication module 330 and provide the usage information of the patient for the rehabilitation training content or information obtained by processing the usage information to the user through the output module 310. The processor 350 may output video information, sensing information, and result information for the rehabilitation training content to the user. Also, the processor 350 may generate a stereoscopic video by applying the usage information of the patient for the rehabilitation training content to the virtual model and provide the generated stereoscopic video to the user through the output module 310. Further, the processor 350 may generate a stereoscopic video by applying preset motion information to the virtual model of the patient and provide the generated stereoscopic video to the user.

The processor 350 may receive setting information for the rehabilitation training content from the user, perform a kinematic simulation of causing the virtual model of the patient to perform the rehabilitation training content to which the received setting information is applied, and provide at least one of a stereoscopic video and a result of the simulation to the user. The processor 350 may check and provide load, rehabilitation training effects, and the like exerted on the patient's body when a specific rehabilitation training is performed, in advance through a simulation employing the digital twin-based virtual model and detect the most appropriate setting of rehabilitation training content for the patient by repeating such a simulation. The processor 350 may receive motion of the user detected through the sensor module 320 as setting information or receive setting information through an input module (e.g., a keyboard, a touchscreen, or the like).

The processor 350 may receive final setting information for the rehabilitation training content from the user through the input module and update setting information for the rehabilitation training content provided to the patient according to the received final setting information. The processor 350 may change the setting information for the rehabilitation training content provided to the patient according to the final setting information set by the user so that more effective rehabilitation training content can be provided to the first user. The processor 350 may access the server 200 or the rehabilitation training provision device through the communication module 330 and change the setting information stored in the corresponding device to update the setting information for the rehabilitation training content.

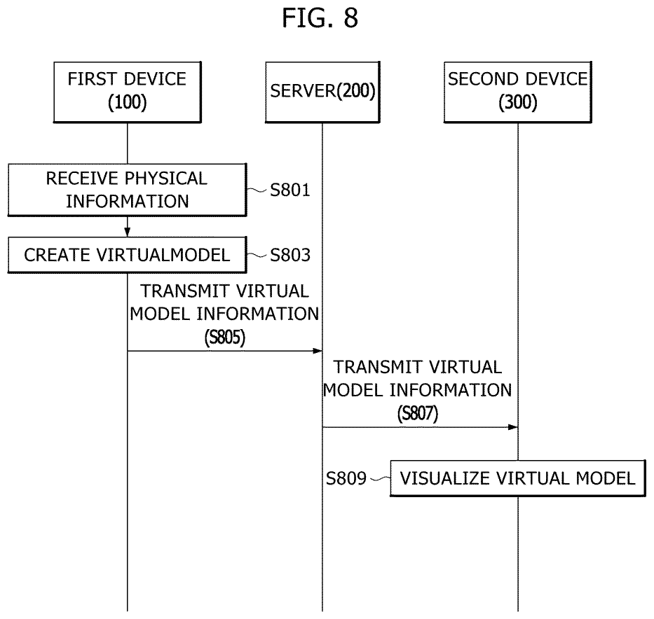

FIG. 8 is a first sequence diagram illustrating a rehabilitation training control method according to an exemplary embodiment of the present invention.

A method of providing a virtual model of a first user to a second user will be described below with reference to FIG. 8. A part of the following process may be omitted or performed in a different order from that described below.

First, the first device 100 may receive physical information of a first user (e.g., a patient) (S801).

Subsequently, the first device 100 may create a virtual model for the first user on the basis of the physical information of the first user using digital twin technology (S803).

Subsequently, the first device 100 may transmit information on the created virtual model to the server 200 through a communication network (S805).

Subsequently, the second device 300 may receive the information on the virtual model of the first user from the server 200 through the communication network (S807).

Subsequently, the second device 300 may visualize the virtual model and provide the visualized virtual model to a second user (e.g., a medical professional) (S809). Here, the second device 300 may generate a stereoscopic video by applying preset motion information to the virtual model and provide the generated stereoscopic video to the second user.

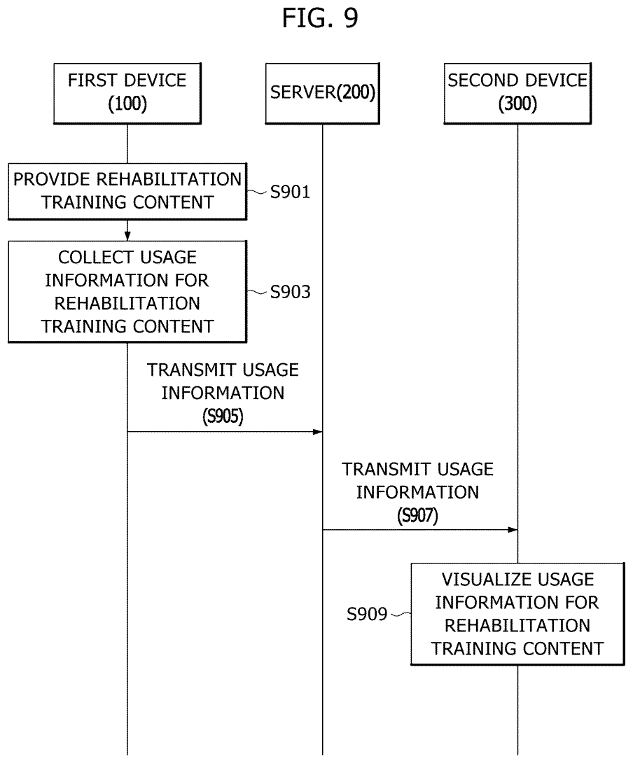

FIG. 9 is a second sequence diagram illustrating a rehabilitation training control method according to an exemplary embodiment of the present invention.

A method of collecting and providing usage information of a first user for rehabilitation training content to a second user will be described below with reference to FIG. 9. A part of the following process may be omitted or performed in a different order from that described below.

First, the first device 100 may provide rehabilitation training content to a first user (e.g., a patient) (S901) and collect usage information of the first user for the rehabilitation content (S903).

Subsequently, the first device 100 may transmit the usage information of the first user for the rehabilitation training content to the server 200 through a communication network (S905).

Subsequently, the second device 300 may receive the usage information of the first user for the rehabilitation training content from the server through the communication network (S907).

The second device 300 may visualize the usage information of the first user for the rehabilitation training content stored in the server 200 and provide the visualized usage information to the second user (S909). Here, the second device 300 may generate a stereoscopic video by applying the usage information of the first user for the rehabilitation training content to a virtual model for the first user and provide the generated stereoscopic video to the second user.

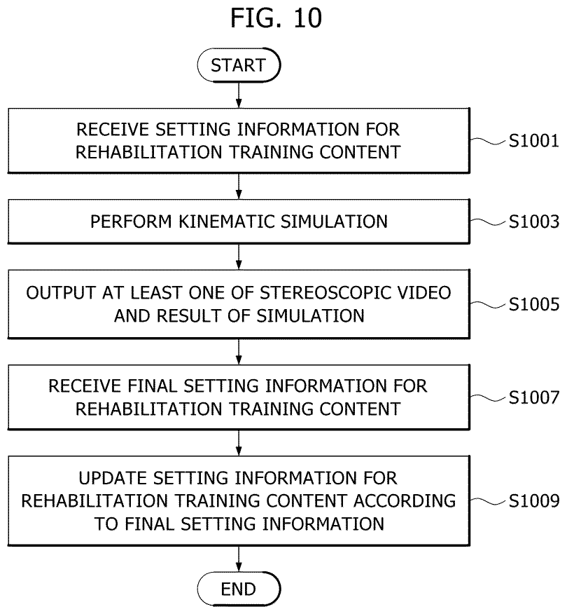

FIG. 10 is a third sequence diagram illustrating a rehabilitation training control method according to an exemplary embodiment of the present invention.

A method of updating setting information for rehabilitation training content provided to a first user will be described below with reference to FIG. 10. A part of the following process may be omitted or performed in a different order from that described below.

First, the second device 300 may receive setting information for rehabilitation training content from a second user (S1001).

Subsequently, the second device 300 may perform a kinematic simulation of causing a virtual model of a first user to perform rehabilitation training content to which the setting information received from the second user is applied (S1003) and provide at least one of a stereoscopic video and a result of the simulation to the second user (S1005). The second user may repeat operations S901 to S905 while changing the setting information for rehabilitation training content.

Subsequently, the second device 300 may receive final setting information for rehabilitation training content from the second user (S1007) and update the setting information for rehabilitation training content provided to the first user according to the received final setting information (S1009).

According to an aspect of the present invention, a patient's information related to usage of rehabilitation training content is shared with a medical professional so that a diagnosis or prescription for the patient can be made remotely.

According to an aspect of the present invention, effects of a patient's rehabilitation training based on a prescription related to rehabilitation training can be predicted through simulation with a medical twin and provided to a medical professional so that the medical professional can properly set a rehabilitation training guide.

Effects of the present invention are not limited to those described above, and other effects which have not been described will be clearly understood by those of ordinary skill in the art from the above descriptions.

Although the present invention has been described above with reference to embodiments illustrated in the drawings, the embodiments are merely illustrative, and those skilled in the art should understand that various modifications and other equivalent embodiments can be made from the embodiments.

Therefore, the technical scope of the present invention should be determined from the following claims.

What is claimed is:

1. A system for controlling rehabilitation of a patient, the system comprising:
a first device configured to create a virtual model for a first user on the basis of physical information of the first user using digital twin technology; and
a second device configured to visualize the virtual model and provide the visualized virtual model to a second user.

2. The system of claim 1, wherein the first device provides rehabilitation training content to the first user and collects usage information of the first user for the rehabilitation training content.

3. The system of claim 2, wherein the usage information includes at least one of video information of the first user, sensing information of the first user, and result information of the rehabilitation training content.

4. The system of claim 3, wherein the second device provides the usage information or information obtained by processing the usage information to the second user.

5. The system of claim 4, wherein the second device generates a stereoscopic video by applying the usage information to the virtual model and provides the stereoscopic video to the second user.

6. The system of claim 2, wherein the second device receives setting information for the rehabilitation training content from the second user, performs a kinematic simulation of causing the virtual model to perform the rehabilitation training content to which the received setting information is applied, and provides at least one of a stereoscopic video and a result of the simulation to the second user.

7. The system of claim 2, wherein the second device receives final setting information for the rehabilitation training content from the second user and updates setting information for the rehabilitation training content provided to the first user according to the final setting information.

8. A method of controlling rehabilitation of a patient which is performed by a patient rehabilitation control system, the method comprising:
creating a virtual model for a first user on the basis of physical information of the first user using digital twin technology; and
visualizing the virtual model and providing the visualized virtual model to a second user.

9. The method of claim 8, further comprising:
providing rehabilitation training content to the first user; and
collecting usage information of the first user for the rehabilitation training content.

10. The method of claim 9, wherein the usage information includes at least one of video information of the first user, sensing information of the first user, and result information of the rehabilitation training content.

11. The method of claim 10, further comprising, after the collecting of the usage information, providing the usage information or information obtained by processing the usage information to the second user.

12. The method of claim 11, wherein the providing of the information obtained by processing the usage information comprises generating a stereoscopic video by applying the usage information to the virtual model and providing the stereoscopic video to the second user.

13. The method of claim 9, further comprising, after the collecting of the usage information:
receiving setting information for the rehabilitation training content from the second user;
performing a kinematic simulation of causing the virtual model to perform the rehabilitation training content to which the received setting information is applied; and
providing at least one of a stereoscopic video and a result of the simulation to the second user.

14. The method of claim 9, further comprising:
receiving final setting information for the rehabilitation training content from the second user; and
updating setting information for the rehabilitation training content provided to the first user according to the final setting information.

15. A device for controlling rehabilitation of a patient, the device comprising:
an output module; and
a processor connected to the output module,
wherein the processor creates a virtual model for a patient on the basis of physical information of the patient using digital twin technology, visualizes the virtual model, and provides the visualized virtual model to a user.

16. The device of claim 15, further comprising a communication module,
wherein the processor receives usage information of the patient for rehabilitation training content through the communication module and provides the usage information or information obtained by processing the usage information to the user through the output module.

17. The device of claim 16, wherein the usage information includes at least one of video information of the patient, sensing information of the patient, and result information of the rehabilitation training content.

18. The device of claim 16, wherein the processor generates a stereoscopic video by applying the usage information to the virtual model and provides the stereoscopic video to the user through the output module.

19. The device of claim 16, wherein the processor receives setting information for the rehabilitation training content from the user, performs a kinematic simulation of causing the virtual model to perform the rehabilitation training content to which the received setting information is applied, and provides at least one of a stereoscopic video and a result of the simulation to the user.

20. The device of claim 16, wherein the processor receives final setting information for the rehabilitation training content from the user and updates setting information for the rehabilitation training content provided to the patient according to the final setting information.
